(19) United States
(12) Patent Application Publication
(10) Pub. No.: US 2008/0044895 A1
(43) Pub. Date: Feb. 21, 2008

(54) FILTER SUPPORT FOR USE WITH A TISSUE CASSETTE FOR COLLECTING AND EMBEDDING CELLS

(75) Inventors: rer. nat Gabriele Wedell, Dresden (DE); Christian Heinzelmann, Stolpen (DE)

Correspondence Address: HESLIN ROTHENBERG FARLEY & MESITI PC, 5 COLUMBIA CIRCLE, ALBANY, NY, 12203, US

(73) Assignee: OXYPHEN AG, Lachen (CH)

(21) Appl. No.: 11/777,508

(22) Filed: Jul. 13, 2007

(30) Foreign Application Priority Data
Aug. 17, 2006 (DE) 102006038613.2

(51) Int. Cl. C12M 1/00

(52) U.S. Cl. 435/308.1

(57) ABSTRACT

A filter support is releasably connectable to a tissue cassette for collecting and embedding cells in paraffin wax. The cassette includes a cylindrical port which extends from a top surface through a bottom surface of the cassette. The filter support has a cylindrical extension to extend over a bottom end of the cylindrical port of the tissue cassette. The filter support is made of a polar polymer.

### FIELD OF THE INVENTION

The present invention relates to a filter support for use with a tissue cassette for collecting and embedding cells in paraffin wax. Such tissue cassettes are used for preparing cells for microscopic examination, and for archiving the cells for molecular and immunologic tests. To this end, cells and tissue fragments are embedded within a solid substrate, such as paraffin wax, to enable them to be cut with a microtome for microscopic evaluation, and to be stored in a stable state.

### BACKGROUND

Many disease processes can only be diagnosed on the basis of histologic or cytologic examination using a light microscope. For instance, while the presence of a tumor can be detected using radiological devices, the determination of whether a tumor is benign or malignant still requires a pathologist's interpretation of the appearance of the cells using light microscopy. Before reaching this stage, however, the tissue sample must first be retrieved, collected, and processed for microscopic examination. A number of techniques are available for retrieving and collecting biopsies or cell samples from a patient. It is of benefit to patients to use minimally invasive techniques for obtaining biopsies or cell samples. For example, small tissue fragments can be obtained from fine needle aspiration biopsy, or by brushing body cavity surfaces accessible through minimally invasive endoscopic techniques. Once retrieved, the cells then need to be processed for microscopy. A variety of processing techniques are known for depositing tissue fragments directly onto a microscope slide.

Another technique, commonly referred to as a cell block preparation, has several advantages over the direct deposition of tissue fragments. The cell block procedure immobilizes cells or small tissue fragments in a solid support, typically paraffin wax. Thin sections of the cell block are then cut with a microtome and the sections mounted onto a microscope slide for examination. The resulting sections from the cell block display diagnostic information in a manner that complements the direct deposition techniques. For example, the architectural arrangements of cells to each other is displayed better in section from a cell block than in directly deposited cells on a microscope slide. Cell blocks also permit important diagnostic molecular and immunological tests to be conducted on the cell samples that would otherwise be difficult or impractical on direct preparations. In addition, cell blocks appear to preserve the cells indefinitely in a convenient manner at room temperature, thereby facilitating biomedical research.

The cell block preparation method requires that the cell fragments be “embedded” in a solid medium, most commonly paraffin wax. “Embedding” requires the following generic steps: (1) all water molecules must be removed from the cells, typically by alcohol (water is miscible with alcohol); (2) all alcohol must then be removed, as well as all fatty substances, and replaced typically by xylene (xylene is miscible with alcohol but not water); (3) the xylene must be removed and replaced with wax (wax is miscible with xylene but not with most alcohols or water); and (4) the cells in molten wax must then be manually organized and hardened on the underside of a tissue cassette so that a section of the wax block with the embedded tissue can be cut using a microtome. The first three of these steps are commonly performed by a “tissue processor,” a machine that circulates alcohol, xylene, and molten wax sequentially in a chamber containing the tissue cassette. Tissue cassettes typically serve the dual purpose of containing the cell sample during the embedding process, and for providing an attachment mechanism for holding the wax block on the microtome machine so that the cell sample subsequently embedded in wax on the bottom surface of the cassette is able to be cut into thin sections.

U.S. Pat. No. 6,913,921 B2 to the University of Massachusetts discloses a method and apparatus that utilizes a flow-through processing and embedding technique for rapidly embedding cells. This flow-through processing maximizes the efficiency of cell recovery and of extractions during embedding, thereby decreasing the amount of cellular sample required, minimizing the amount of time for processing, and minimizing the amount of reagents needed for embedding. The method and apparatus also automatically places cells at the plane in the cell block where they need to be sectioned without diluting them with carrier substances.

According to an exemplary embodiment, the apparatus includes a modified tissue cassette that serves a dual function of capturing the cell sample and also providing a pathway through which the embedding reagents can flow. A cell flow pathway is provided for delivering cell fragments from a collected cell sample, typically in either an aqueous solution or a liquid cell-preservative, through the modified tissue cassette to a filter that traps the cells and tissue fragments. The apparatus also includes a reagent flow pathway configured to sequentially pass embedding reagents, then lastly paraffin, through the tissue cassette and through the cell sample deposited on the filter. When the paraffin is cooled, the filter is peeled away from the paraffin embedded cells, and a gasket between the filter and the tissue cassette is pulled away from the tissue cassette. This technique leaves a disk of wax protruding from the tissue cassette with the embedded cells placed at the plane at which a tissue section can be cut using a standard microtome.

Referring to the accompanying drawings of U.S. Pat. No. 6,913,921 B2, the tissue cassette 30 includes a cylindrical port 36 which extends from a top surface 32 through a bottom surface 34 of the cassette 30. The cylindrical port 36 creates a flow-through pathway for the cells and reagents to be delivered. The tissue cassette 30 is designed to provide sufficient structural rigidity to eliminate the need for wax to fill the entire cassette 30, thereby conserving paraffin. The tissue cassette 30 should be constructed from a material which is resistant to degradation from alcohol, xylene or acids, as is allegedly the case for polypropylene, polypropylene with talc, and nylon. The cylindrical port 36 extending out of the bottom surface 34 of the tissue cassette 30 is removably connected to a flat, ring-shaped, gasket 42. The gasket 42 approximately matches the diameter and thickness of the wall of the cylindrical port 36 of the tissue cassette 30 and it can be composed of a pliable material such as Viton™ rubber that allows the gasket 42 to form a water-tight seal with the tissue cassette 30 and resists chemical degradation upon exposure to xylene and other embedding reagents.

The gasket 42 is removably connected to a filter 40 that is larger in diameter than the inner diameter of the gasket 42. The filter 40 has a pore size in the range of about 6-12 microns in diameter to allow sub-cellular debris to flow through to the filter 40 while trapping small-sized individual cells. The filter 40 can comprise a polycarbonate filter (such as, e.g., isopore™ made by Millipore corporation, Billerica, Mass.) which are smooth and prevent the cells and the wax from sticking to the filters as they are pulled away. In addition, the gasket 42, tissue cassette 30 and filter 40 can be lightly bonded together so that these three components can be quickly loaded onto the apparatus 10.

The filter 40 rests on a filter support 60. A water-tight seal can be formed between the cylindrical port 36 of the tissue cassette 30 and the filter support 60, with the filter 40 itself sandwiched therebetween. The filter support 60 can have a cylindrical extension to extend over the gasket 42 and bottom end of the cylindrical port 36 of the tissue cassette 30. With this configuration, a correct seating of the tissue cassette 30 with respect to the filter support 60, gasket 42, and filter 40 can be assured. The filter support 60 can include integrated heating and cooling elements in order to maintain the paraffin in a melted state and to speed up the paraffin hardening process.

The filter support 60 is removably connected to a waste container 50 for capturing the waste fluid during the embedding process, which includes fluid media containing the cell sample, and the excess embedding reagents.

While the described method and apparatus have entailed great advantages for the processing of cell samples, it has been found that removing the filter support from the cassette without deforming the wax disk protruding from the bottom surface of the cassette is a challenging task due to the strong bond between the paraffin wax and the filter support. It is therefore an object of the present invention to make improvements to the apparatus described in U.S. Pat. No. 6,913,921 B2 so as to make removal of the filter support from the cassette easier.

### SUMMARY OF THE INVENTION

The present invention provides a filter support for use with a tissue cassette which enables easy removal of the filter support from the paraffin wax. The following two approaches have been found to be successful in reaching this aim:

It is well known that polar substances can be dissolved using polar solvents, while non-polar substances require non-polar solvents. Polarity, as used herein, shall mean the property of compounds which describes how equally bonding electrons are shared between atoms and which affects other physical properties such as solubility, melting and boiling point. Polarity affects intermolecular forces, leading to some compounds or molecules within compounds being labelled as polar or non-polar. The degree of polarity of a compound's molecules depends on the difference in electronegativity between atoms in its molecules and the asymmetry of the compound's structure A non-polar molecule has no net dipole. This can be achieved by the molecule having only non-polar bonds or polar bonds arranged symmetrically so that the dipoles cancel out. A polar molecule has a net dipole. This is achieved by the molecule being made up of polar bonds arranged unsymmetrically so that the dipoles do not cancel out. Water (H₂O) and table salt (NaCl) are prominent examples of polar compounds, while ammonia (NCl₃) and methane (CH₄) are examples of non-polar compounds. With regard to the solubility of compounds, it is a general rule that “like solves like”.

Extensive experiments on the applicant's part have led to the result that this relationship is also true with regard to the strength of a bond between two different materials brought into close contact with each other, as may be the case when molten material A is filled into a mold made of material B. Here, the general rule could be expressed as “like bonds like”. In other words, it has been found that for two given substances, the strength of the adhesive bond between them depends on the degree of similarity of their respective polarities. Therefore, a polar substance will develop a strong bond with another polar substance, while the bond will be weaker if the other substance is less polar or non-polar. Likewise, a non-polar substance will develop a strong bond with another substance that is of low polarity or non-polar, while it is less adhesive to a polar substance.

The discovery of this behavior of materials, combined with the knowledge that paraffin waxes commonly used for embedding cells are nonpolar in the sense described above, has led to the inventive concept of manufacturing the filter support from a polar material. Since the filter support is a one-way, disposable part, it must be produced in large numbers at low cost. Therefore, it is advisable to select an appropriate polar polymer as the material for the filter support. This has a further advantage in that most polymers are suitable for making bodies of complex geometry.

Known polar polymers which can advantageously be used for manufacturing the filter support include polyester, polycarbonate, polyamide and polyimide. It will be understood that the invention is not limited to these polymers but can be carried out successfully by using any polar polymer.

It is also advantageous to give at least the lower section of the inner surface of the cylindrical extension of the filter support a conical shape which acts as a draw, i.e. a mold release slope, inside the cylindrical extension. The conical shape makes removal of the filter support from the wax disk protruding from the bottom surface of the cassette easier. At the same time, if the upper section of the inner surface of the cylindrical extension of the filter support is also given a conical shape, a conical joint between the cylindrical extension of the filter support and the conical outer surface of the cylindrical port of the cassette forms, resulting in a firm and liquid-tight connection between them and making removal of the filter support from the cylindrical port of the cassette easier. The liquid-tight connection thus established has a further advantage in that no additional gasket is needed.

Another approach that has proven to be successful in making removal of the filter support from the cassette easier is to provide the filter support with a mechanical aid therefor. This approach has led to the inventive concept of providing the filter support with means for applying a loosening force between the cassette and the filter support.

In a first embodiment, the means for applying a loosening force comprises an internal thread in the cylindrical extension of the filter support. In this case, the cylindrical port of the cassette must of course be equipped with a corresponding external thread. In this embodiment, the filter support would be screwed on and off the bottom end of the cylindrical port of the cassette. However, due to the twisting motion involved in this process, deformations of the wax disk cannot be completely ruled out unless measures are taken to ensure low adhesion between the paraffin wax and the filter support. Such a measure could, for instance, be to make the filter support from a polar polymer, as described above.

In a preferred embodiment of the invention, the means for applying a loosening force comprises at least one knee lever. The knee lever, as used herein, shall mean a lever that comprises of two distinct lever sections, which form an angle between them. For instance, the angle may be 90°, resulting in an L-shaped lever. The knee lever is arranged on the upper edge of the filter support, so as to be pivotally supported thereon. Thus, when the first lever section of the knee lever is actuated, the second lever section exerts pressure, i.e. a loosening force, on the cassette, resulting in the filter support disengaging from the cassette and the wax disk protruding from its underside. If more than one knee lever is used, then it is beneficial to arrange the knee levers symmetrically with respect to the cylindrical extension in order to effect uniform action of the respective loosening forces.

Especially in the case where the material of the filter support is a polymer, the filter support and the knee lever or knee levers can advantageously be formed as a one-piece part, for example by arranging each knee lever such that the second lever section is connected to the edge of the cylindrical extension and extends away from this edge, i.e. parallel to the bottom surface of the cassette, and that the first lever section, beginning from the outer end of the second lever section, extends parallel to the cylindrical extension, i.e. away from the bottom surface of the cassette. If the connection between the knee lever and the cylindrical extension of the filter support is sufficiently thin, then this connection acts as a hinge between the cylindrical extension and the knee lever about which the knee lever can be pivoted. To disconnect the filter support from the cassette, a force, exerted on the free end of the first section of the knee lever and directed away from the cylindrical extension of the filter support, will result in the second section of the knee lever exerting a loosening force on the bottom surface of the cassette. This can be done manually.

It is also advantageous to give at least the upper section of the inner surface of the cylindrical extension of the filter support a conical shape which makes it interengageable with a conical outer surface of the bottom end of the cylindrical port of a tissue cassette. The conical shape helps form a conical joint between the cylindrical port of the cassette and the cylindrical extension of the filter support, resulting in a firm and liquid-tight connection between them, which makes the use of a gasket dispensable. Furthermore, removal of the filter support from the cylindrical port of the cassette is made easier. At the same time, if the lower section of the inner surface of the cylindrical extension of the filter support is also given a conical shape, this section acts as a draw, i.e. a mould release slope inside the cylindrical extension of the filter support. The conical shape makes removal of the filter support from the wax disk protruding from the bottom surface of the cassette easier.

It is to be understood that it is especially beneficial to combine features from both concepts for best results, i.e. to decrease the bonding forces between the paraffin wax and the filter support by choosing an appropriate filter support material as described in the first inventive concept and, at the same time, to provide the filter support with means for applying a loosening force between the cassette and the filter support as described in the second inventive concept.

### BRIEF DESCRIPTION OF THE DRAWINGS

The invention can be more fully understood from the following detailed description, taken in conjunction with the accompanying drawings in which:

FIG. 1 shows in a longitudinal sectional view (from top to bottom) a tissue cassette, a filter support, a support disk, the top part of a waste receptacle and an assembly containing all these items.

FIG. 2 shows (from left to right) a longitudinal sectional view and a front elevational view of the tissue cassette and filter support during the process of removing the filter support from the cassette.

### DETAILED DESCRIPTION OF THE DRAWINGS

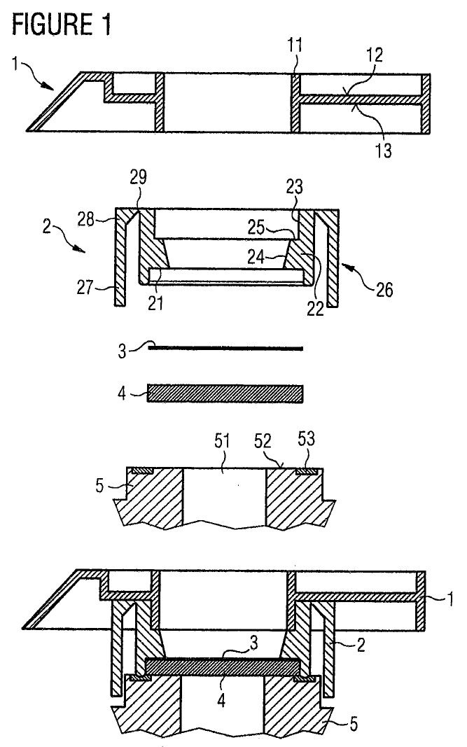

Referring to FIG. 1, the depicted tissue cassette 1 for collecting and embedding cells in paraffin wax includes a cylindrical port 11 which extends from a top surface 12 through a bottom surface 13 of the tissue cassette 1. The cylindrical port 11 provides a cell flow pathway for delivering cell fragments from a collected cell sample through the tissue cassette 1 to a filter 3 that traps the cells and tissue fragments. The filter 3 is a thin membrane which is supported in a filter support 2. The filter support 2 comprises a support disk seat 21 with a cylindrical extension 22 and a support disk 4 which is attached to the support disk seat 21. The cylindrical extension 22 is adapted to fit over the bottom end of the cylindrical port 11 of the tissue cassette 1.

The filter 3, which is a thin membrane, is sandwiched between the support disk 4 and the support disk seat 21 of the filter support 2 such that the filter 3 covers the upper surface of the support disk 4 completely. In the embodiment shown in the drawings, the support disk 4 is made of sintered bronze granules. The support disk 4 is sufficiently rigid to support the filter 3, yet porous enough to let reagents flow through. The cylindrical extension 22 of the filter support 2 as well as the filter 3 are made of polycarbonate. Polycarbonate is a plastic material of high polarity. Since the paraffin wax used to embed the cell samples is of low polarity, it will develop a very weak bond with the filter and the filter support after curing.

The cylindrical extension 22 has an inner surface which comprises an upper section 23 which is designed to fit over the lower end of the cassette's cylindrical port 11 and a lower section 24 which will, when in use, shape the wax block containing the embedded cells. Both sections of the inner surface of the cylindrical extension 22 of the filter support 2 are conical, i.e. they have tapered walls. The upper section 23 has a very small slope so that it forms a liquid-tight connection with the cylindrical port 11 of the tissue cassette 1 without running the risk of the filter support 2 falling off due to its own deadweight. The lower section 24 of the inner surface is conical with a slope greater than that of the upper section 23 so that easy removal of the filter support 2 from the wax block is promoted. Due to the conical shape of the lower section 24 of the inner surface, the wax block protruding from the bottom surface 13 of the tissue cassette 1 will be of conical shape as well.

Furthermore, the smallest diameter of the upper section 23 is greater than the biggest diameter of the lower section 24, so that a shoulder 25 is formed between the upper section 23 and the lower section 24. This shoulder 25 serves as a stop surface which limits the translatory displacement of the filter support 2 when attaching it to the cylindrical port 11 of the tissue cassette 1. By providing this stop surface 25 it is made sure that the relative positions of the tissue cassette 1 and the filter support 2 are well-defined.

Two identical knee levers 26 are arranged symmetrically on the upper edge of the cylindrical extension 22 of the filter support 2, i.e. in diametric opposition to each other with respect to the circular cross section of the cylindrical extension 22. The knee levers 26 act as a means for applying a loosening force upon the tissue cassette 1. Each of the two knee levers 26 comprises two lever sections. The relatively long first lever section 27 is needed to operate the knee levers 26 and it extends substantially parallel to the cylindrical extension 22. The relatively short second lever section 28 of each knee lever 26 extends substantially perpendicularly between the upper edge of the cylindrical extension 22 and the first lever section 27 of the knee lever 26, thereby connecting the first lever section 27 to the cylindrical extension 22. Adjacent the cylindrical extension 22, the cross-sectional area of the second lever section 28 is relatively small so as to enable a swiveling movement of the knee lever 26 by making it bendable about this thinned spot 29. The knee levers 26 and the filter support 2 are formed unitarily, i.e., the knee levers 26 and the cylindrical extension 22 form a one-piece part.

For the purpose of explaining the function of the filter support 22, the top part of a waste receptacle 5 is also shown. The waste receptacle 5 has an opening 51 to collect the reagents used in the embedding process after they have passed the filter 3 and the support disk 4. The opening 51 is surrounded by a contact surface 52 upon which the filter support 2 is mounted when in use. The contact surface 52 has a circular groove containing a gasket 53, which is made of an elastic material.

During the preparation of a cell sample, the items described above are assembled in the following way, as can be seen in the lowermost drawing of FIG. 1:

The support disk 4 is mounted in the support disk seat 21 of the filter support 2 such that the filter 3 is sandwiched between the support disk seat 21 and the support disk 4. The filter support 2 is connected to the tissue cassette 1, such that the cylindrical extension 22 extends over the bottom end of the cylindrical port 11 of the tissue cassette 1. The filter support 2, together with the tissue cassette 1, is placed on the contact surface 52 of the waste receptacle 5 such that the edge of the support disk seat 21 is in direct contact with the gasket 53 held in the contact surface 52, thus establishing a liquid-tight connection between them.

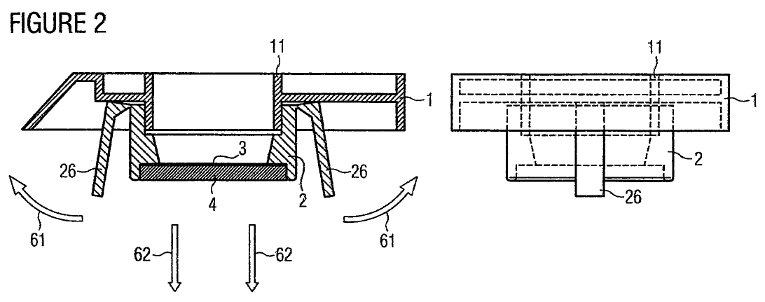

Referring now to FIG. 2, the process of removing the filter support 2 from the tissue cassette 1 will be explained in more detail. Upon completion of the curing process of the paraffin wax, the filter support 2 must be removed from the tissue cassette 1 in order to expose the wax block for microtome cutting. To this end, the operator simply has to put two fingers on the free ends of both knee levers 26 and then exert outwardly directed forces on them. This may, of course, also be done using a specialised tool or inside an automated apparatus, as the case may be. In the drawing, these forces are represented by the arched arrows 61.

The resulting pivotal movement of the knee levers 26 has the effect that the respective second lever sections 28 of both knee levers 26 will be pressed against the bottom surface 13 of the tissue cassette 1. The loosening force thus applied will disconnect the filter support 2, together with the filter 3 and the support disk 4, from the tissue cassette 1 and the wax block. The downwardly directed arrows 62 in the drawing represent the downward displacement of the filter support 2 caused by the loosening forces. The choice of materials described above will promote the removal since there will be only little adhesion between the paraffin wax and the filter 3 and cylindrical extension 22 of the filter support 2, respectively.

1. A filter support releasably connectable to a tissue cassette for collecting and embedding cells in paraffin wax, the cassette including a cylindrical port which extends from a top surface through a bottom surface of the cassette and the filter support having a cylindrical extension to extend over a bottom end of the cylindrical port of the tissue cassette, wherein the filter support is made of a polar polymer.

2. The filter support of claim 1, wherein the polar polymer is selected from the group consisting of polyester, polycarbonate, polyamide or polyimide.

3. The filter support of claim 1, wherein the filter support comprises means for applying a loosening force between the cassette and the filter support.

4. The filter support of claim 3, wherein the means for applying a loosening force comprises an internal thread in the cylindrical extension of the filter support.

5. The filter support of claim 3, wherein the means for applying a loosening force comprises at least one knee lever which is pivotally supported on the cylindrical extension.

6. The filter support of claim 5, wherein at least two knee levers are arranged symmetrically with respect to the cylindrical extension.

7. The filter support of claim 5, wherein the filter support and the knee lever or knee levers are a one-piece part.

8. The filter support of claim 1, wherein at least a lower section of an inner surface of the cylindrical extension of the filter support is conical so as to act as a mould release slope inside the cylindrical extension, enabling easier removal of the filter support from the cassette and a wax disk protruding from the bottom surface.

9. The filter support of claim 1, wherein at least an upper section of an inner surface of the cylindrical extension of the filter support is conical so as to be interengageable with a conical outer surface of the bottom end of the cylindrical port of the tissue cassette.

10. A filter support releasably connectable to a tissue cassette for collecting and embedding cells in paraffin wax, the cassette including a cylindrical port, which extends from a top surface through a bottom surface of the cassette and the filter support having a cylindrical extension to extend over a bottom end of the cylindrical port of the tissue cassette, wherein the filter support comprises means for applying a loosening force between the cassette and the filter support.

11. The filter support of claim 10, wherein the means for applying a loosening force comprises an internal thread in the cylindrical extension of the filter support.

12. The filter support of claim 10, wherein the means for applying a loosening force comprises at least one knee lever which is pivotally supported on the cylindrical extension.

13. The filter support of claim 12, wherein at least two knee levers are arranged symmetrically with respect to the cylindrical extension.

14. The filter support of claim 12, wherein the filter support and the knee lever or knee levers are a one-piece part.

15. The filter support of claim 10, wherein the filter support is made of a polar polymer.

16. The filter support of claim 15, wherein the polar polymer is selected from the group consisting of polyester, polycarbonate, polyamide or polyimide.

17. The filter support of claim 10, wherein at least a lower section of an inner surface of the cylindrical extension of the filter support is conical so as to act as a mould release slope inside the cylindrical extension, enabling easier removal of the filter support from the cassette and a wax disk protruding from the bottom surface.

18. The filter support of claim 10, wherein at least an upper section of an inner surface of the cylindrical extension of the filter support is conical so as to be interengageable with a conical outer surface of the bottom end of the cylindrical port of a tissue cassette.
